(19) United States
(12) Patent Application Publication
(10) Pub. No.: US 2011/0274644 A1
(43) Pub. Date: Nov. 10, 2011

(54) LENS CARE COMPOSITIONS

(76) Inventor: Kasey Jon Minick, Cumming, GA (US)

(21) Appl. No.: 13/182,749

(22) Filed: Jul. 14, 2011

Related U.S. Application Data
(63) Continuation of application No. 11/599,976, filed on Nov. 15, 2006.
(60) Provisional application No. 60/737,215, filed on Nov. 16, 2005.

(51) Int. Cl. A61K 31/79; A61P 27/02

(52) U.S. Cl. 424/78.36

(57) ABSTRACT

The present invention provides a lens care composition having a persistent lipid removal efficacy characterized by that the amount of lipids adsorbed by a silicone hydrogel lens after about 10 cycles of soiling with a lipid soiling solution and subsequent cleaning with a lens care solution of the invention under no-rubbing conditions is about 70% or less of that after about 10 cycles of soiling with the lipid soiling solution and subsequent cleaning with an aqueous phosphate buffer under no-rubbing conditions. The lens care composition comprises at least one surfactant, a polyvinylpyrrolidone (PVP), xylitol, a buffering agent, wherein in combination with polyvinylpyrrolidone (PVP) and xylitol, the surfactant provides the persistent lipid removal efficacy.

This application claims the benefit under 35 U.S.C. §119 (e) of U.S. provisional application Ser. No. 60/737,215 filed Nov. 16, 2005.

This invention relates generally to aqueous solutions useful for treating contact lenses. Such solutions are particularly useful as a basis for formulating contact lens care products.

### BACKGROUND OF THE INVENTION

In recent years, multiple-purpose solutions (MPSs), which clean, disinfect, and rinse contact lenses all without mechanically rubbing lenses, have been developed as a new type of lens care systems. Cleaning usually refers to removal of lipids, proteins or other matter which has become affixed to a lens. Disinfecting usually refers to inactivating of harmful bacteria or fungi whenever the lenses are removed from the eye, which is usually on a daily basis. Rinsing usually refers to removing debris from the lens before placing the lens in the eye. These new systems start dominating the most of the lens care market. Such popularity is most likely derived from the easiness and convenience provided by these new systems to consumers. Because lenses typically are directly used without rinsing after being treated with a MPS, it is desirable that a MPS would exhibit virtually non-existent cytotoxicity, very low irritation, efficiently antimicrobial activity, and good cleaning efficacy and could provide comfort to lens wearers.

However, currently available MPSs may not possess all of the desired properties listed above. For example, current MPSs may not have a good efficacy in removing lipids from a worn lens, in particular silicone hydrogel contact lenses which are highly susceptible to lipid deposition and adsorption. Especially after many cycles of wearing and cleaning with a MPS, lipids may accumulate so greatly to adversely affect the performance of a lens (e.g., visual acuity, comfort, etc.). Furthermore, without mechanically rubbing worn lenses, their cleanness would be difficult to be maintained (or preserved) as clean as new lenses. Deposits (e.g., proteins and/or lipids, the likes) on contact lenses will decrease their light transmissibility (or transmittance) and thereby affect adversely visual acuity which the lenses can provide to an user.

Therefore, there is a need for developing a multipurpose lens care solution which has a good efficacy in removing lipids from worn lenses, non-existent cytotoxicity, very low irritation, efficiently antimicrobial activity.

### SUMMARY OF THE INVENTION

Generally described, the present invention provides a lens care composition which comprises at least one surfactant, a polyvinylpyrrolidone (PVP), and xylitol, wherein the combination of polyvinylpyrrolidone (PVP), xylitol and the surfactant provides the persistent lipid-removal efficacy. The composition of the invention allows for the formulation of a multipurpose lens care solution for disinfecting, cleaning, and rinsing contact lens without rubbing lenses while preserving their cleanness as substantially clean as new lenses.

The present invention provides the foregoing and other features, and the advantages of the invention will become further apparent from the following detailed description of the example embodiments set forth herein, read in conjunction with the accompanying FIGURES. The detailed description and FIGURES are merely illustrative of the invention and do not limit the scope of the invention, which is defined by the appended claims and equivalents thereof.

### BRIEF DESCRIPTION OF THE DRAWINGS

FIG. 1 shows the amount of lipids adsorbed by a silicone hydrogel contact lens (AcuVue® Advance™) as function of number of cycle of simulated wearing and cleaning with a lens care solution without rubbing.

### DETAILED DESCRIPTION OF EXAMPLE EMBODIMENTS

The present invention may be understood more readily by reference to the following detailed description of the invention taken in connection with the accompanying drawing FIGURES, which form a part of this disclosure. Unless defined otherwise, all technical and scientific terms used herein have the same meaning as commonly understood by one of ordinary skill in the art to which this invention belongs. Generally, the nomenclature used herein is well known and commonly employed in the art. Conventional methods are used for carrying out the disclosed procedures, such as those provided in the art and various general references. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, reference to singular forms such as “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The invention relates to a lens care composition having a persistent lipid-removal efficacy characterized by having at most 70% of an amount of lipids adsorbed by a silicone hydrogel lens (AcuVue® Advance™) after about 10 cycles of lipid soiling and subsequent cleaning with a phosphate buffer. The present invention is based upon the unexpected and beneficial finding that a formulation containing at least a surfactant, a polyvinylpyrrolidone (PVP), and xylitol can have a persistent lipid removal efficacy. When such formulation is used to clean contact lenses (AcuVue® Advance™) soiled with a lipid aqueous solution comprising FITC-Phosphatidylethanolamine (FITC-PE, from Molecular Probes) at a concentration of about 0.5 μg/ml (equivalent to physiological lipid concentration in tears) (pH ˜7.0), it removes effectively lipids from the soiled lenses. Especially, the amount of lipids adsorbed by a silicone hydrogel lens (AcuVue® Advance™) decreases or at least remains substantially constant after 5 cycle of soiling and cleaning with a solution of the invention, whereas the amount of lipids adsorbed by a silicone hydrogel lens (AcuVue® Advance™) increases after 5 cycle of soiling and cleaning with a phosphate buffer or with Optifree® Express lens MPS disinfecting solution (from Alcon), as the number of cycle of soiling and cleaning increases.

A lens care composition of the invention can be used to clean contact lenses including hard (PMMA) contact lenses, soft (hydrophilic) contact lenses, and rigid gas permeable (RGP) contact lenses. The soft contact lenses are hydrogel contact lens or preferably silicone hydrogel contact lenses.

A “hydrogel” refers to a polymeric material which can absorb at least 10 percent by weight of water when it is fully hydrated. Generally, a hydrogel material is obtained by polymerization or copolymerization of at least one hydrophilic monomer in the presence of or in the absence of additional monomers and/or macromers.

A “silicone hydrogel” refers to a hydrogel obtained by copolymerization of a polymerizable composition comprising at least one silicone-containing vinylic monomer or at least one silicone-containing macromer.

“Hydrophilic,” as used herein, describes a material or portion thereof that will more readily associate with water than with lipids.

The term “cleaning” means that the solution contains one or more active ingredients in sufficient concentrations to loosen and remove loosely held lens deposits and other contaminants on the surface of the article to be cleaned. While not necessary with the present invention, a user may wish to use the solutions of the present invention in conjunction with digital manipulation (for example, manual rubbing of the lens with a solution) or with an accessory device that agitates the solution in contact with the lens, for example, a mechanical cleaning aid.

In accordance with the invention, a lens care composition is ophthalmic safe. The term “ophthalmically safe” with respect to a lens care solution is meant that a contact lens treated with the solution is safe for direct placement on the eye without rinsing, that is, the solution is safe and sufficiently comfortable for daily contact with the eye via a contact lens. An ophthalmically safe solution has a tonicity and pH that is compatible with the eye and comprises materials, and amounts thereof, that are non-cytotoxic according to international ISO standards and U.S. FDA regulations.

The term “compatible with the eye” means a solution that may be in intimate contact with the eye for an extended period of time without significantly damaging the eye and without significant user discomfort.

A “cycle of lipid soiling and subsequent cleaning with a solution” is intended to describe a process composed of a soiling step and then a cleaning step, in which the soiling step is performed by immersing a silicone hydrogel lens (AcuVue® Advance™) in 1 ml of FITC-Phosphatidylethanolamine (FITC-PE) solution (0.5 μg/ml) for about 16 hours @37° C., the cleaning step is performed by immersing the lipid-soiled silicone hydrogel lens (AcuVue® Advance™) in 1 ml of a cleaning solution or a phosphate buffer for about 8 hours. This cycle simulates a cycle of lens wearing and subsequent lens cleaning with a lens care solution.

A “persistent lipid removal efficacy” in reference to a lens care solution is intended to describe that the lens care solution is still efficient to remove lipids adsorbed by a silicone hydrogel lens (AcuVue® Advance™) even after at least 5 cycles of lipid soiling and cleaning with it. The persistent lipid removal efficacy is preferably characterized by that the amount of lipids adsorbed by a silicone hydrogel lens (AcuVue® Advance™) after about 10 cycles of soiling with a lipid soiling solution and subsequent cleaning with the lens care solution under no-rubbing conditions is about 70% or less of that after about 10 cycles of soiling with the lipid soiling solution and subsequent cleaning with an aqueous phosphate buffer under no-rubbing conditions, wherein the lipid soiling solution is an aqueous solution comprises FITC-Phosphatidylethanolamine at a concentration of 0.5 μg/ml. The procedures for performing soling and cleaning cycles and for determining the amount of lipids adsorbed by a lens are described in Example 6.

In one aspect, the invention provides a lens care composition comprising at least one surfactant, a polyvinylpyrrolidone (PVP), and xylitol, wherein in the combination of with polyvinylpyrrolidone (PVP), Xylitol and the surfactant provides the persistent lipid-removal efficacy, wherein the persistent lipid removal efficacy is characterized by that the amount of lipids adsorbed by a silicone hydrogel lens (AcuVue® Advance™) after about 10 cycles of soiling with a lipid soiling solution and subsequent cleaning with the lens care composition under no-rubbing conditions is about 70% or less of that after about 10 cycles of soiling with the lipid soiling solution and subsequent cleaning with an aqueous phosphate buffer under no-rubbing conditions, wherein the lipid soiling solution is an aqueous solution comprises FITC-Phosphatidylethanolamine at a concentration of 0.5 μg/ml.

Any suitable known surfactants can be used in the invention. Examples of suitable surfactants include, but are not limited to poloxamers under the tradename Pluronic from BASF Corp. (Pluronic™ and Pluronic-R™) which are nonionic surfactants consisting of block copolymers of propylene oxide and ethylene oxide; poloxamine which is a block copolymer derivative of ethylene oxide and propylene oxide combined with ethylene diamine; tyloxapol, which is 4-(1,1,3,3-tetramethylbutyl)phenol polymer with formaldehyde and oxirane; ethoxylated alkyl phenols, such as various surface active agents available under the tradenames TRITON (Union Carbide, Tarrytown, N.Y., USA) and IGEPAL (Rhone-Poulenc, Cranbury, N.J., USA); polysorbates such as polysorbate 20, including the polysorbate surface active agents available under the tradename TWEEN (ICI Americas, Inc., Wilmington, Del., USA.); alkyl glucosides and polyglucosides such as products available under the tradename PLANTAREN (Henkel Corp., Hoboken, N.J., USA); and polyethoxylated castor oils commercially available from BASF under the trademark CREMAPHOR.

Preferred surfactants include homopolymers of polyethylene glycol or polyethyleneoxide, and certain poloxamers such as materials commercially available from BASF under the tradenames PLURONIC® 17R4, PLURONIC® F-68NF, PLURONIC® F68LF, and PLURONIC® F127, with PLURONIC® F-68NF (National Formulary grade) being the most preferred. When present, poloxamers may be employed at from about 0.001% to about 5% by weight, preferably from about 0.005% to about 1% by weight, more preferably from about 0.05% to about 0.6% by weight.

The polyvinylpyrrolidone (PVP) used in the compositions of the invention is a linear homopolymer or essentially a linear homopolymer comprising at least 90% repeat units derived from 1-vinyl-2-pyrrolidone monomers, the polymer more preferably comprising at least about 95% or essentially all of such repeat units, the remainder selected from polymerization-compatible monomers, preferably neutral monomers, such as alkenes or acrylates. Other synonyms for PVP include povidone, polyvidone, 1-vinyl-2-pyrolidinone, and 1-ethenyl-2-pyrolionone (CAS registry number 9003-39-8). The PVP used in the present invention suitably has a weight average molecular weight of about 10,000 to 250,000, preferably 30,000 to 100,000. Such materials are sold by various companies, including ISP Technologies, Inc. under the trademark PLASDONE™ K-29/32, from BASF under the trademark KOLLIDON™ for USP grade PVP, for example KOLLIDON™ K-30 or K-90. While the invention is not limited to any specific PVP, K-90 PVP is preferred, more preferably pharmaceutical grade.

In accordance with the invention, polyvinylpyrrolidone can function as a lubricant and provide comfort to an eye. Polyvinylpyrrolidone can be present up to 2% by weight, preferably from about 0.1% to about 1.0% by weight, more preferably from about 0.2% to about 0.5% by weight.

Xylitol is a five-carbon sugar alcohol that is found naturally in many plants and fruits. It has been used as a sweetener in food products such as chewing gum because it is noncaloric and has a sweetness quality equal to that of sugar. Xylitol can be used as a tonicity agent to adjust the tonicity (osmolality) of a lens care composition. Xylitol is used in a preferred contact lens care composition of the invention in an amount of from about 0.4% to about 10% by weight, more preferably in an amount of from about 1.0% to about 8% by weight, most preferably in an amount of from 2% to about 6% by weight, based on the total amount of contact lens care composition which is advantageously formulated in aqueous solution.

It has now surprisingly been found that the combination PVP and xylitol with at least one surfactant can possesses an enhanced lipid removal efficacy. This guards against the appearance of dryness, which can lead to a reduced lachrymal film. The usage of the above-described active ingredient combination can also substantially improves comfort when wearing contact lenses. Negative effects caused by surface-active substances and preservatives are reduced and the contact lenses are prevented from drying out.

In a preferred embodiment, the lens care solution is a multipurpose solution capable of disinfecting, cleaning, and rinsing a contact lens.

The term “disinfecting solution” means a solution containing one or more microbiocidal compounds, that is effective for reducing or substantially eliminating the presence of an array of microorganisms present on a contact lens, which can be tested by challenging a solution or a contact lens after immersion in the solution with specified inoculums of such microorganisms. The term “disinfecting solution” as used herein does not exclude the possibility that the solution may also be useful for a preserving solution or that the disinfecting solution may additionally be useful for daily cleaning, rinsing, and storage of contact lenses.

A solution that is useful for cleaning, chemical disinfection, storing, and rinsing an article, such as a contact lens, is referred to herein as a “multi-purpose solution.” Such solutions may be part of a “multi-purpose solution system” or “multi-purpose solution package.” The procedure for using a multi-purpose solution, system or package is referred to as a “multi-functional disinfection regimen.” Multi-purpose solutions do not exclude the possibility that some wearers, for example, wearers particularly sensitive to chemical disinfectants or other chemical agents, may prefer to rinse or wet a contact lens with a another solution, for example, a sterile saline solution prior to insertion of the lens. The term “multi-purpose solution” also does not exclude the possibility of periodic cleaners not used on a daily basis or supplemental cleaners for removing proteins, for example enzyme cleaners, which are typically used on a weekly basis.

A disinfecting solution of the invention can be used to disinfect contact lenses against a wide range of microorganisms including but not limited to Fusarium solani, Staphylococcus aureus, Pseudomonas aeruginosa, Serratia marcescens and Candida albicans. For the purposes of the present invention the term “disinfect” means the rendering non-viable of substantially all pathogenic microbes that are in the vegetative state, including gram negative and gram positive bacteria, as well as fungi. The chemical compounds and compositions that render such pathogenic microbes inactive are known as microbicides.

A disinfecting or MPS solution of the invention must contain a microbicide in a concentration sufficient to effect the desired disinfection of a contact lens. The specific concentrations required for the microbicides useful in this invention must be determined empirically for each microbicide. Some of the factors affecting the effective concentration are specific activity of the microbicide against the specified pathogens, the molecular weight of the microbicide, and the solubility of the microbicide. It is also important that the chosen microbicides be employed in a physiologically tolerable concentration. The list of microbicides which may be employed in the present invention include, but is not in limited to biguanides, biguanide polymers, salts thereof, N-alkyl-2-pyrrolidone, polyquaternium-1, bronopol, benzalkonium chloride, and hydrogen peroxide. The presently useful antimicrobial biguanides include biguanides, biguanide polymers, salts thereof, and mixtures thereof. Preferably, the biguanide is selected from alexidine free-base, salts of alexidine, chlorhexidine free-base, salts of chlorhexidine, hexetidine, hexamethylene biguanides, and their polymers, and salts thereof. Most preferably, the biguanide is a hexamethylene biguanide polymer (PHMB), also referred to as polyaminopropyl biguanide (PAPB).

Typical solutions of this invention contain the microbicides PHMB in an amount of from about 0.01 to about 10 ppm, preferably from about 0.05 to about 5 ppm, more preferably from about 0.1 to about 2 ppm, even more preferably from about 0.2 to about 1.5 pp.

Although PHMB has a broad spectrum of activity and non-specific mode of action against bacteria, PHMB might be able to cause some level of corneal staining (Jones Lyndon, et. al. “Asymptomatic corneal staining associated with the use of balafilcon silicon-hydrogel contact lenses disinfected with a polyaminopropyl biguanide—preserved care regimen”, Optometry and Vision Science 79: 753-61 (2002)). Therefore, it would be desirable to lower the amount of PHMB in a lens care solution while maintaining the antimicrobial efficacy of the lens care solution. It has been shown in studies that the addition of PVP, a cellulose ether and xylitol does not have negative effects on the antimicrobial efficacy of a disinfecting solutions but could increase the microbiological efficacy of PHMB present in the contact lens care compositions according to the invention without resulting in negative effects as regards toxicity. The concentration of PHMB can be reduced to about 0.5 ppm.

Where a lens care composition comprises a biguanide or a biguanide polymer (PHMB) as a microbiocide, it comprises preferably less than 1000 ppm, more preferably less than 500 ppm, even more preferably less than 100 ppm chloride ions. A 0.6% sodium chloride solution, which is probably close to the concentration of sodium chloride in eye, would result in almost 3600 ppm chloride ions in the solution. Such a high concentration of chloride ion would diminish the antimicrobial effectiveness of PHMB, especially those having less than 0.5 ppm PHMB.

The present compositions preferably include an effective amount of a chelating component. Any suitable, preferably ophthalmically acceptable, chelating component may be included in the present compositions, although ethylenediaminetetraacetic acid (EDTA), salts thereof and mixtures thereof are particularly effective. EDTA is low level non-irritating chelating agent and can be synergistic with PHMB to increase antimicrobial efficacy. Typical amount of EDTA is from about 0.001% to about 1% by weight, preferably from about 0.002% to about 0.5% by weight, more preferably from about 0.004% to about 0.1, even more preferably from about 0.005 to about 0.05, based on the total amount of contact lens care composition.

The composition of the present invention preferably contains a buffering agent. The buffering agents maintain the pH preferably in the desired range, for example, in a physiologically acceptable range of from about 6.3 to about 7.8, preferably between 6.5 to 7.6, even more preferably between 6.8 to 7.4. Any known, physiologically compatible buffering agents can be used. Suitable buffering agents as a constituent of the contact lens care composition according to the invention are known to the person skilled in the art. Examples are boric acid, borates, e.g. sodium borate, citric acid, citrates, e.g. potassium citrate, bicarbonates, e.g. sodium bicarbonate, TRIS (trometamol, 2-amino-2-hydroxymethyl-1,3-propanediol), bis-aminopolyols, phosphate buffers, e.g. Na₂HPO₄, NaH₂PO₄, and KH₂PO₄ or mixtures thereof. The amount of each buffer agent is that amount necessary to be effective in achieving a desired pH of the composition. Typically, it is present in an amount of from 0.001% to 2%, preferably from 0.01% to 1%; most preferably from about 0.05% to about 0.30% by weight.

The preferred buffering agents are bis-aminopolyols of formula (I)

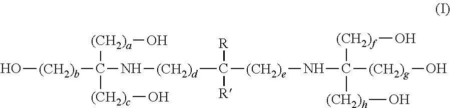

wherein a, b, c, d, e, f, g, and h are independently an integer from 1 to 6; and R and R′ are independently selected from the group consisting of —H, —CH₃, —(CH₂)₂₋₆—H, and —(CH₂)₁₋₆—OH. In the present invention, the buffering agents described by formula (I) may be provided in the form of various water-soluble salts. A most preferred bis-aminopolyol is 1,3-bis(tris[hydroxymethyl]methylamino)propane (bis-TRIS-propane).

It has been found that bis-TRIS-propane can exhibit a synergy with certain microbicides (e.g., PHMB) and fungicides, resulting in a microcidal activity significantly higher than the activity of these same active ingredients used in conjunction with other buffers. BIS-TRIS propane is described under biological buffers in Biochemicals and Reagents, Sigma-Aldrich Co., 2000-2001 edition. The specific structure of bis-TRIS-propane is shown in formula II.

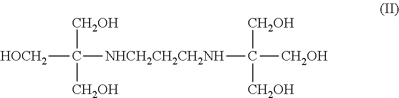

The dissociation constants for this dibasic compound are pKa₁=6.8 and pKa₂=9.5 which renders aqueous solutions of this compound useful as a buffering agent in a broad pH range from about 6.3 to 9.3. bis-TRIS-propane at a concentrations used in this invention is harmless to the eye and to known contact lens materials and is, therefore, ophthalmically compatible.

Preferably, the solutions of the present invention have a low concentration of phosphate ions, preferably substantially free of phosphate ions. Solutions having less than a total of 1500 ppm of phosphate ion and chloride ion have been surprisingly discovered to be effective against a broad spectrum of microorganisms, including C. albicans. Previously known solutions generally had very high concentrations of both phosphate ions and chloride ions, due to their use large amounts of phosphate buffers, sodium or potassium chloride tonicity agents, and hydrochloric or phosphoric acid to adjust pH downward.

The solutions of the present invention optionally can contain dexpanthenol. Dexpanthenol is an alcohol of pantothenic acid, also called Provitamin B5, D-pantothenyl alcohol or D-panthenol. Dexpanthenol may be used in the solutions according to the invention in an amount of 0.005% to 10%, especially in an amount of 0.01 to 5%, preferably in an amount of 0.01 to 1%, more preferably in an amount of 0.01 to 0.5%, most preferably from about 0.01 to 0.25%.

Apart from the above-described ingredients, a contact lens care composition of the invention generally contain one or more other constituents, e.g. ocularly acceptable tonicity agents (substances that affect the tonicity) other than xylitol, viscosity-enhancing agent, etc. Although it is generally unnecessary, an enzymatic cleaning substance may also be present in the contact lens care products according to the invention. The amounts of these or other conventional additives used in the contact lens care compositions according to the invention are variable within the limits known to the person skilled in the art.

The contact lens care products typically are formulated in such a way that they are isotonic with the lachrymal fluid. A solution which is isotonic with the lachrymal fluid is generally understood to be a solution whose concentration corresponds to the concentration of a 0.9% sodium chloride solution. Deviations from this concentration are possible throughout, provided that the contact lenses to be treated are not damaged.

The isotonicity with the lachrymal fluid, or even another desired tonicity, may be adjusted by adding xylitol and optionally organic or inorganic substances which affect the tonicity. Suitable occularly acceptable tonicity agents include, but are not limited to sodium chloride, potassium chloride, glycerol, mannitols, sorbitol, and mixtures thereof. The tonicity of the solution is typically adjusted to be in the range from about 200 to about 450 milliosmol (mOsm), preferably from about 200 to 450 mOsm, preferably from about 250 to 350 mOsm.

In accordance with the invention, the solutions of the present invention optionally can contain a viscosity enhancing agent which is preferably a cellulose ether, more preferably methyl cellulose (MC), ethyl cellulose, hydroxymethylcellulose, hydroxyethyl cellulose (HEC), hydroxypropylcellulose, hydroxypropylmethyl cellulose (HPMC), or a mixture thereof. Even more preferably, a cellulose ether is hydroxyethyl cellulose (HEC), hydroxypropylmethyl cellulose (HPMC), or a mixture thereof. The cellulose ether is present in the composition in an amount of from about 0.01% to about 5% by weight, preferably from about 0.05% to about 3% by weight, even more preferably from about 0.1% to about 1% by weight, based on the total amount of contact lens care composition. It is believed that a cellulose ether can be used to increase the viscosity of a lens care and also can serve as a lubricant in the lens care composition.

The solutions are compatible with both hard and soft type lenses, and are adaptable for use with virtually any of the commonly known disinfecting techniques, including “cold” soaking under ambient temperature conditions, as well as with high temperature disinfecting methods. The disinfecting solutions of the present invention are especially noteworthy for their wide spectrum of bactericidal and fungicidal activity at low concentrations coupled with very low toxicity and reduced affinity for binding and concentrating when used with soft type contact lenses.

The contact lens care compositions according to the invention are suitable for all kinds of contact lenses. This includes in particular the so-called hard and soft contact lenses, and also the so-called hard-flexible or highly gas-permeable contact lenses. The contact lens care compositions according to the invention have cleaning action and, in addition, optionally have antimicrobial action.

The contact lens care compositions according to the invention are produced in known manner, in particular by means of conventional mixing of the constituents with water or dissolving the constituents in water.

Aqueous solutions comprising the following components have been found to be particularly useful in cleaning and disinfecting contact lenses:

| | |
|---|---|
| poloxamer | 0.005% to 1% |
| PVP | 0.01% to 1% |
| Xylitol | 1% to 8% |
| PHMB | less than 1.5 ppm |
| EDTA | less than 0.1% |
| Bis-TRIS-propane | 0.001% to 2% |

Even more preferred are those solutions having the following components:

| | |
|---|---|
| poloxamer | 0.05% to 0.4% |
| PVP | 0.05% to 0.5% |
| Xylitol | 2% to 6% |
| PHMB | less than 1.2 ppm |
| EDTA | 0.001% to 0.006% |
| Bis-TRIS-propane | 0.05% to about 0.30% |

The compositions according to the invention are especially suitable for cleaning and, where appropriate, for disinfecting contact lenses. The contact lens care compositions according to the invention are used in known manner, e.g. by bringing the contact lens into contact with the contact lens care composition for a period of time that is sufficient to clean or disinfect it. Depending on the lens type and the degree of soiling, a sufficient time span ranges from a few minutes to about 24 hours, preferably from about 1 to about 12 hours, more preferably from about 2 to about 8 hours, even more preferably from about 4 to about 12 hours, has proved to be practicable.

The contacting temperature is in the range preferred from about 0° C. to about 100° C., more preferably from about 10° C. to about 60° C., still more preferably from about 15° C. to about 37° C. Contacting at or about ambient temperature is very convenient and useful. The contacting preferably occurs at or about atmospheric pressure.

Where a lens care solution is a multipurpose solution, the contacting preferably occurs for a time in the range of from about 5 minutes or about 1 hour to about 12 hours or more. Especially preferred are those solutions have 0.5 ppm or less PHMB and can obtain at least a 1 log reduction in C. albicans within 15 minutes of contact with the lens. Also preferred are those having less than 0.25 ppm PHMB and obtaining at least 1.0, more preferably 1.5 log, reduction in C. albicans within 15 minutes, more preferably at least a 2.0 log reduction in C. albicans within 30 minutes.

The contact lens can be contacted with the solution by immersing the lens in the solution. Although not necessary, the solution containing the contact lens can be agitated, for example, by shaking the container containing the solution and contact lens, to at least facilitate removal of deposit material from the lens.

In another aspect, the invention provides a method for cleaning and/or disinfecting contact lenses. The method comprises the step of bringing one or more contact lenses into contact with the contact lens care composition of the invention for a period of time that is sufficient to clean and/or disinfect the one or more contact lenses.

The solutions and methods of the present invention may be used in conjunction with enzymes to remove debris or deposit material from the contact lens as the solutions of the present invention have no negative effect on the proteolytic activity of enzymes, such as UNIZYME®. After such contacting step, the contact lens optionally may be manually rubbed with saline, or even rinsed without rubbing, to remove further deposit material from the lens. The cleaning method can also include rinsing the lens substantially free of the liquid aqueous medium prior to returning the lens to a wearer's eye.

In a further aspect, the present invention provides a kit for cleaning and/or disinfecting contact lenses. The kit comprises a bottle containing a lens care solution, wherein the lens care solution can be dispensed from the bottle into a container where the lens care solution is in contact with one or more contact lenses for a period of time sufficient long to clean and/or disinfect them, wherein the lens care solution comprises at least one surfactant, a polyvinylpyrrolidone (PVP), xylitol, and a buffering agent, wherein in combination with polyvinylpyrrolidone (PVP) and xylitol, the surfactant provides the persistent lipid-removal efficacy, wherein the persistent lipid removal efficacy is characterized by that the amount of lipids adsorbed by a silicone hydrogel lens (AcuVue® Advance™) after about 10 cycles of soiling with a lipid soiling solution and subsequent cleaning with the lens care solution under no-rubbing conditions is about 70% or less of that after about 10 cycles of soiling with the lipid soiling solution and subsequent cleaning with an aqueous phosphate buffer under no-rubbing conditions, and wherein the lipid soiling solution is an aqueous solution comprises FITC-Phosphatidylethanolamine at a concentration of 0.5 μg/ml.

The kit can optionally include one or more lens care cases for treating contact lenses and/or instructions for how to use the lens care solution to clean and/or disinfect contact lenses.

The previous disclosure will enable one having ordinary skill in the art to practice the invention. In order to better enable the reader to understand specific embodiments and the advantages thereof, reference to the following examples is suggested.

### EXAMPLE 1

Aqueous solutions are prepared to have the compositions shown in Tables 1a and 1b in purified water.

TABLE 1a

| Formulation No. | 1 | 2* | 3* | 4* | 5* | 6 |
|---|---|---|---|---|---|---|
| PHMB (ppm) | 1.0 | 1.0 | 1.0 | 1.0 | 1.0 | 1.0 |
| EDTA (%) | 0.004 | 0.004 | 0.004 | 0.004 | 0.004 | 0.004 |
| Sorbitol (%) | 2.50 | | | 4.50 | 4.50 | |
| Xylitol (%) | 2.0 | 4.0 | 3.4 | | | 2.9 |
| NaH₂PO₄ | | 0.30 | 0.46 | 0.30 | 0.46 | |
| Bis Tris Propane (%) | 0.141 | | | | | 0.141 |
| TRIS Tromethamine) (%) | | 0.166 | 0.332 | 0.166 | 0.332 | |
| Pluronic F127 (%) | | | | 0.10 | 0.10 | 0.10 |
| Pluronic F87 (%) | 0.100 | 0.100 | 0.100 | | | |
| Pluronic 17R4 (%) | | | | | | |
| Tyloxopol (%) | 0.02 | 0.02 | 0.02 | 0.02 | 0.02 | |
| Kollidon 90F (PVP K-90) (%) | 0.2 | 0.2 | 0.2 | 0.2 | 0.2 | 0.2 |
| Dexpanthenol (%) | 0.02 | 0.02 | 0.02 | 0.02 | 0.02 | 0.02 |
| 5N HCl (%) | 0.141 | | | | | 0.141 |
| pH | 7.20 | 7.07 | 7.25 | 7.01 | 7.29 | 7.04 |
| Osmolality (mOsm) | 301 | 328 | 320 | 314 | 342 | 211 |

*Adjusted pH to 7.0-7.3 with 8N H₃PO₄ (%)

TABLE 1b

| Formulation No. | 7 | 8 | 9 | 10 | 11 | 12 |
|---|---|---|---|---|---|---|
| PHMB (ppm) | 1.0 | 1.0 | 1.0 | 1.0 | 1.0 | 1.0 |
| EDTA (%) | 0.004 | 0.004 | 0.004 | 0.004 | 0.004 | 0.004 |
| Sorbitol (%) | | 3.65 | 3.65 | | | |
| Xylitol (%) | 2.9 | | | 2.9 | 2.9 | 3.0 |
| NaH₂PO₄ | | | | | | |
| Bis Tris Propane | 0.141 | 0.141 | 0.141 | 0.141 | 0.141 | 0.141 |
| TRIS (%) (Tromethamine) | | | | | | |
| Pluronic F127 (%) | 0.10 | 0.10 | 0.10 | 0.10 | 0.10 | 0.10 |
| Pluronic F87 (%) | | | | | | |
| Pluronic 17R4 (%) | | | | 0.10 | 0.05 | 0.05 |
| Tyloxopol (%) | | | | | | |
| Kollidon 90F (PVP K-90) (%) | 0.2 | 0.2 | 0.2 | 0.2 | 0.2 | 0.2 |
| Dexpanthenol (%) | | 0.02 | | 0.02 | 0.02 | |
| 5N HCl (%) | 0.141 | 0.141 | 0.141 | 0.141 | 0.141 | 0.139 |
| pH | 6.98 | 6.98 | 7.04 | 7.02 | 7.03 | 7.14 |
| Osmolality (mOsm) | 213 | 208 | 223 | 215 | 213 | 221 |

### EXAMPLE 2

This example illustrates various tests for cytotoxicity of a lens care solution prepared in Example 1.
alimarBlue™ Reduction Assay. Cell viability in the presence of a lens care solution prepared in Example 1 is assayed according to the alamarBlue™ reduction assay procedure from BioSource International, Inc. Either L929 cells or HCE-T cells are used in the tests. A testing mixture is prepared by diluting a lens care solution with an equal volume of the growth medium (50% dilution). alamarBlue™ reduction is then measured fluorometrically at 24 hours.
Neutral Red Based Assay. Cell viability in the presence of a lens care solution prepared in Example 1 is assayed according to the neutral red based in vitro toxicology assay from SIGMA®. Either L929 cells or HCE-T cells are used in the tests. A testing mixture is prepared by diluting a lens care solution with an equal volume of the growth medium (50% dilution). Absorbance at around 540 nm is measured spectrophotometrically at 24 hours.
USP Elution Test. Cytotoxicity of a lens care solution is evaluated by using the Standard USP Elution Test (“Biological Reactivity Tests, in vitro: Elution Test”, USP). A lens care solution is diluted with serum-supplemented cell culture medium at 25% test solution concentration. Each culture is examined microscopically after 48 hours using trypan blue for the presence of morphological changes, reduction in cell density or cell lysis induced by the test solution. Solutions 10-12 prepared in Example 1 all pass the USP elution tests.
ISO Ocular Irritation Study. Solution 12 prepared in Example 1 is tested for its irritation to ocular tissue of the rabbit according to ISO Ocular Irritation Study protocol. No irritating effect is found with Solution 12.

The results of cell viability in the presence of a lens care solution of the invention are shown in Table 2.

TABLE 2

| | Cell Viability | | | |
|---|---|---|---|---|
| [symbol] | Test 1 | Test 2 | Test 3 | Test 4 |
| 1 | 69.69 | 50.50 | N/A | N/A |
| | | (Borderline Cytotoxic) | | |
| 2 | 60.79 | 34.65 | N/A | N/A |
| | | (Cytotoxic) | | |
| 2* | 72.12 | 62.97 | 85.01 | 35.82 |
| | | | | (Cytotoxic) |
| 3 | 53.24 | 37.14 | N/A | N/A |
| | | (Cytotoxic) | | |
| 3* | 70.10 | 61.63 | 79.88 | 36.36 |
| | | | | (Cytotoxic) |
| 4 | 82.68 | 85.99 | N/A | N/A |
| 5 | 68.00 | 62.18 | N/A | N/A |
| 6 | 92.22 | 81.40 | 95.56 | 89.87 |
| 7 | 93.80 | 79.76 | 93.58 | 84.54 |
| 8 | 94.59 | 61.82 | 96.83 | 92.32 |
| 9 | 96.22 | 61.49 | 95.32 | 96.91 |
| 10 | 93.71 | 69.67 | 96.20 | 101.52 |
| 11 | 93.77 | 73.93 | 94.68 | 98.51 |
| 12 | 93.77 | 76.50 | 101.57 | 105.17 |

*Repeated tests with new Tris.
Test 1. Alimar Blue (AB) and L929 Cells @ 24 Hrs Exposure;
Test 2. Neutral Red Uptake (NRUR) and L929 Cells @ 24 Hrs Exposure;
Test 3. Alimar Blue (AB) and HCE-T Cells @ 24 Hrs Exposure;
Test 4. Neutral Red Uptake (NRUR) and HCE-T Cells @ 24 Hrs Exposure. [symbol]
indicates data missing or illegible when filed

### EXAMPLE 3

A series of tests are conducted to evaluate the disinfecting performance (microbial efficacy) of solutions prepared in accordance with Example 1 against Fusarium solani (F. solani), Candida albicans (C. albicans), Serratia marcescens (S. marcescens), Staphylococcus aureus (S. aureus), and Pseudomonas aeruginosa (P. aeruginosa). Inoculation levels for each test are between 1.0×10⁵ and 1.0×10⁶ cfu/ml. For each organism, at least two inoculums are used and the obtained results are averaged as shown in Table 3.

TABLE 3

| Formulation | S. aureus | | C. albicans | | S. marcescens | | F. solani | | P. aeruginosa | |
|---|---|---|---|---|---|---|---|---|---|---|
| Number | 5 Min | 4 Hr | 5 Min | 4 Hr | 5 Min | 4 Hr | 5 Min | 4 Hr | 5 Min | 4 Hr |
| 1 | 3.4 | >5.2 | 4.0 | >5.0 | 3.1 | >5.2 | 1.4 | >5.0 | 4.3 | >5.2 |
| 2 | 3.2 | 5.1 | 2.1 | >5.0 | 3.3 | >5.2 | 0.8 | 3.2 | 2.9 | >5.2 |
| 2* | 4.1 | >5.2 | 1.5 | 4.2 | 3.6 | >5.0 | 0.5 | 3.2 | 4.5 | >5.3 |
| 3 | 2.8 | 4.8 | 0.6 | 2.4 | 2.6 | >5.2 | 0.3 | 1.9 | 2.7 | >5.2 |
| 3* | 3.8 | >5.2 | 1.0 | 3.2 | 3.9 | >5.0 | 0.5 | 3.4 | 4.5 | >5.3 |
| 4 | 3.3 | >5.2 | 2.4 | >5.0 | 3.5 | >5.2 | 0.8 | 3.5 | 3.7 | >5.2 |
| 5 | 3.0 | >5.2 | 1.2 | 4.6 | 3.5 | >5.2 | 0.8 | 3.5 | 3.8 | >5.2 |
| 6 | 3.2 | >5.3 | 3.1 | >5.1 | 1.1 | 1.2 | 1.4 | >4.7 | >5.2 | >5.2 |
| 7 | 3.5 | >5.3 | 3.0 | >5.1 | 1.0 | 0.0 | 0.9 | >4.7 | >5.2 | >5.2 |
| 8 | 4.2 | >5.3 | 2.8 | >5.1 | 1.2 | 1.2 | 0.8 | >4.7 | >5.2 | >5.2 |
| 9 | 4.0 | >5.3 | 2.9 | >5.1 | 1.1 | 0.2 | 0.9 | >4.7 | >5.2 | >5.2 |
| 10 | 2.2 | 4.3 | 1.3 | >5.1 | 0.8 | 0.7 | 0.1 | >4.7 | >5.2 | >5.2 |
| 11 | 2.2 | >5.3 | 1.4 | >5.1 | 0.8 | 0.9 | 0.3 | >4.7 | >5.2 | >5.2 |
| 12 | 3.6 | >5.3 | 3.2 | >5.1 | 3.7 | >5.3 | 0.2 | >5.2 | 5.4 | >5.4 |

### EXAMPLE 4

A series of tests are conducted to evaluate the disinfecting performance (microbial efficacy) of some solutions prepared in accordance with Example 1 against Fusarium solani (F. solani), Candida albicans (C. albicans), Serratia marcescens (S. marcescens), Staphylococcus aureus (S. aureus), and Pseudomonas aeruginosa (P. aeruginosa) after being stored at 80° C. for an extended period of time. Inoculation levels for each test are between 1.0×10⁵ and 1.0×10⁶ cfu/ml. For each organism, at least two inoculums are used and the obtained results are averaged as shown in Table 4.

TABLE 4

| Formulation | S. aureus | | C. albicans | | S. marcescens | | F. solani | | P. aeruginosa | |
|---|---|---|---|---|---|---|---|---|---|---|
| Number | 5 Min | 4 Hr | 5 Min | 4 Hr | 5 Min | 4 Hr | 5 Min | 4 Hr | 5 Min | 4 Hr |
| 1 ^{a} | 1.1 | 5.2 | 3.1 | >5.0 | 2.9 | >5.0 | 0.8 | >5.0 | 3.8 | >5.2 |
| 1 ^{b} | 1.0 | >5.2 | 3.1 | >5.0 | 3.0 | >5.0 | 0.9 | 5.0 | 4.2 | >5.2 |
| 4 ^{a} | 2.4 | >5.2 | 1.5 | 4.0 | 3.1 | >5.0 | 0.4 | 2.4 | 3.3 | >5.2 |
| 4 ^{b} | 1.7 | >5.2 | 1.4 | 4.1 | 3.2 | >5.0 | 0.3 | 2.2 | 3.4 | >5.2 |
| 5 ^{a} | 2.3 | 5.2 | 0.7 | 3.1 | 3.2 | >5.0 | 0.7 | 2.6 | 3.5 | >5.2 |
| 5 ^{b} | 2.2 | >5.2 | 1.0 | 3.1 | 3.3 | >5.0 | 0.6 | 2.4 | 3.1 | >5.2 |
| 12 ^{c} | 2.0 | >5.3 | 2.6 | >5.0 | 2.5 | 5.2 | 0.9 | >4.9 | >5.3 | >5.3 |
| 12 ^{a} | 1.5 | 4.3 | 2.3 | >5.0 | 2.4 | 5.2 | 0.4 | >4.9 | 4.2 | >5.3 |
| 12 ^{b} | 1.2 | >5.3 | 1.9 | 5.0 | 2.3 | >5.2 | 0.5 | >4.9 | 4.3 | >5.3 |

^{a} stored at 80° C. for 12 days;
^{b} stored at 80° C. for 16 days;
and ^{c} stored at 80° C. for 8 days.

### EXAMPLE 5

A series of tests are conducted to evaluate the compatibility of the solution prepared in Example 1 with CIBASOFT® (CIBA Vision), FOCUS® 1-2 WEEK LENSES® (CIBA Vision), FreshLook®, (CIBA Vision), FOCUS® NIGHT & DAY® (CIBA Vision), O2OPTIX™ (CIBA Vision), AcuVue® 2, AcuVue® Advance™, PureVision™. It is found that, after repeated treatments of those lenses with the solution, there is no significant difference in lens parameters such as, for example, diameter, base curve, and center thickness. The solution is compatible with the tested lenses.

### EXAMPLE 6

This example illustrates a procedure for testing the lipid-removing efficacy of a lens care solution (i.e., the capability of a lens care-solution in removing lipids from lenses. Lenses under study will be divided into three groups: test lenses; control lenses; and standard lenses. Test lenses are first soaked in a solution fluorescently-labeled lipid (e.g., FITC-Phosphatidylethanolamine (FITC-PE) from Molecular Probes from Molecular Probes) for a period of time and then soaked in a lens care solution for another period time. Control lenses are soaked in phosphate buffer (PBS) and not treated with a lens care solution. Standard lenses are soaked in a solution of FITC-PE at a known concentration for establishing a standard curve for determining FITC-PE contration. The experimental procedure is as follows:
1. Equilibrate lenses each in 1 ml PBS in one of the wells of a 24-well polystyrene plates overnight.
2. Using 24 well polystyrene plates, soak each test lens in one well with 1 ml of 0.5 μg/ml (equivalent to physiological concentration) FITC-Phosphatidylethanolamine (FITC-PE) from Molecular Probes for 16 hours @37° C. Soak each control lens (the same lenses) in 1 ml PBS. The numbers of test and control lenses are preferably five.
3. A standard curve plate should also be prepared on the same day by soaking one lens (identical to the test and control lenses) in a well containing FITC-Phosphotidylethanolamine at one of the concentrations ranging from 0-1 ug/ml. (e.g., 1, 0.5, 0.25, 0.125, 0.0625 and 0 ug/ml). Preferably, two lenses are used at each concentration point. Incubate these lenses @37° C.
4. After the 16 hour soak, rinse both test and control lenses 3 times each using 1 ml PBS. Transfer all lenses to the wells of a 24 well plate each well containing 1 ml PBS for each lens and measure fluorescence using a plate reader (e.g., Wallac). These lenses will be referred to as Day 0 lenses. Run the standard curve plate as well. The ‘standard curve plate’ is performed on the day of any given sample/control plate run. Lenses in the ‘standard curve plate’ is not rinsed or transferred, but remains in the original wells.
5. Transfer all the test lenses into 24 well plates containing 1 ml of a lens-care solution for 8 hours. Control lenses will be soaked in PBS for the same 8 hour period. These will be allowed to sit on the bench top.
6. Rinse each lens 3 times each using 1 ml PBS then transfer to a fresh plate containing PBS. Measure fluorescence of both sample/control plates and the standard curve plate. These will be identified as Day 1 lenses.
7. Place lenses into wells each containing 1 ml of fresh 0.5 ug/ml FITC-PE solution (for test lenses) or 1 ml of fresh PBS (for control lenses). Let these lenses soak overnight (16 hours) @37° C. and continue such cycling over a period of 5 days, 7 days, or 2 weeks.
8. Fluorescence readings will be obtained on Days 0, 1, 5, 7, 10 and 14. Calculate lipid concentrations based on the specific standard curve readings. Compare lipid uptake on the lens groups after cleaning with a lens-care solution over a cycling period.

### EXAMPLE 7

Lipid-removal efficacy of a lens care solution (solution 12) is studied in comparison with Alcon's Optifree® Express by no rub regime and with phosphate buffer. Commercially available silicon hydrogel contact lenses, Acuvue® Advance™ are used.

Alcon's Optifree® Express MPS disinfecting solution contains citrate, Tetronic 1304 (tetra-functional block copolymers based on ethylene oxide and propylene oxide), AMP-95 (Aminomethyl Propanol), sodium chloride, boric acid, sorbitol, AMP-95, edetate Disodium, 0.001% Polyquad®** (polyquaternium-1), and 0.0005% Aldox®** (myristamidopropyl dimethylamine).

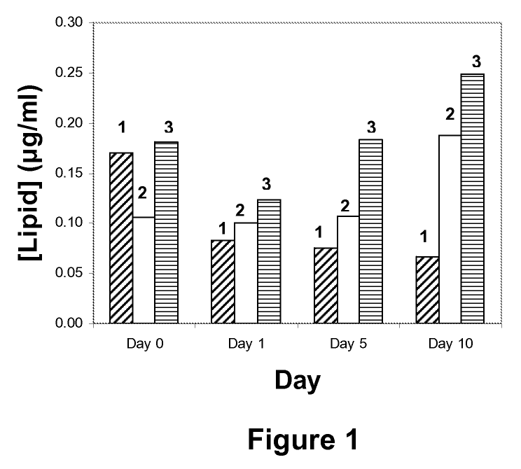

Lipid-removal efficacy study is performed according to the procedure described in Example 6. The results are shown in FIG. 1. Columns 1 represent the lipid concentrations in the lens before and after a number of cycle (lipid soaking and cleaning with solution 12); Columns 2 represent the lipid concentrations in the lens before and after a number of cycle (lipid soaking and cleaning with Optifree® Express MPS disinfecting solution); Columns 3 represent the lipid concentrations in the lens before and after a number of cycle (lipid soaking and cleaning with phosphate buffer). The results indicate that a lens care solution of the invention has a persistent cleaning-efficacy better than Alcon's Optifree® Express MPS disinfecting solution in removing lipids. The solution of the invention can keep lipid concentration in lens at a level much lower than than Optifree® Express MPS disinfecting solution does after 10 cycles of soiling (simulated lens usage) and cleaning. When using the solution of the invention to clean lenses, the in-lens lipid level remain substantially constant or decreased slightly. In contrast, when using Alcon's Optifree® Express MPS disinfecting solution to clean lenses, the in-lens lipid level increases gradually as the number of cycles of soiling (simulated lens usage) and cleaning increases.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part. Furthermore, titles, headings, or the like are provided to enhance the reader's comprehension of this document, and should not be read as limiting the scope of the present invention. Accordingly, the spirit and scope of the appended claims should not be limited to the description of the preferred versions contained therein.

1-23. (canceled)

24. An aqueous contact lens care solution, comprising:
at least one nonionic surfactant, a polyvinylpyrrolidone (PVP), xylitol, Bis- Tris-Propane and a PHMB,
wherein the xylitol is present in an amount of from about 1% to about 8% by weight, wherein the polyvinylpyrrolidone is present in an amount of from about 0.1% to about 1% by weight,
wherein the Bis- Tris-Propane is present in an amount of 0.001% to 2%, wherein the PHMB is present in an amount of less than 1.5 ppm,
wherein the lens care solution has a about 0.3 log more reduction in 5 minutes against C. albicans than an aqueous contact lens care solution having substantially identical composition except of replacing xylitol with sorbitol.

25. The aqueous contact lens care solution of claim 24, wherein the at least one surfactant is a nonionic surfactant consisting of block copolymers of propylene oxide and ethylene oxide, wherein the polyvinylpyrrolidone is a linear homopolymer comprising at least 90% repeat units derived from 1-vinyl-2-pyrrolidone monomers.

26. The aqueous contact lens care solution of claim 25, wherein the at least one surfactant is present in an amount of from about 0.005% to about 1% by weight.

27. The aqueous contact lens care solution of claim 25, wherein the concentration of said PHMB is from about 0.05 to about 5 ppm.

28. The aqueous contact lens care solution of claim 27, wherein the concentration of said PHMB is from about 0.1 to about 2 ppm.

29. The aqueous contact lens care solution of claim 28, wherein the concentration of said PHMB is less than 1.5 ppm, where the aqueous contact lens care solution comprises less than 1000 ppm chloride ions.

30. The aqueous contact lens care solution of claim 29, wherein said solution has a tonicity of from about 200 to about 450 milliosmol (mOsm).

31. The aqueous contact lens care solution of claim 29, further comprising a chelating agent.

32. The aqueous contact lens care solution of claim 31, wherein said chelating agent is EDTA.
